(19) United States
(12) Patent Application Publication
(10) Pub. No.: US 2012/0295332 A1
(43) Pub. Date: Nov. 22, 2012

(54) SYSTEMS AND METHODS FOR DELIVERING OXYGEN TO A VESSEL

(76) Inventor: Alan T. Cheng, Naperville, IL (US)

(21) Appl. No.: 13/475,163

(22) Filed: May 18, 2012

Related U.S. Application Data
(60) Provisional application No. 61/487,800, filed on May 19, 2011.

(51) Int. Cl. C12M 1/06; C12N 1/00; C12N 1/14; C12N 1/20; C12N 1/16

(52) U.S. Cl. 435/252.1; 435/255.1; 435/254.1; 435/243; 435/296.1

(57) ABSTRACT

Oxygen is delivered into a liquid contained within a vessel. Oxygen-rich gas is introduced at an inner mixing region of a vessel toward an agitating element for dissolving some of the oxygen into the liquid. Air is introduced to the liquid at a location different from where the oxygen-rich gas is introduced, minimizing coalescing of bubbles of the oxygen-rich gas and air bubbles. Running the agitating element in the vessel may induce mixing of the liquid and improve oxygen dissolution. Movement of the air bubbles can also generate a mixing effect on the liquid, yet independent from mixing caused by the agitating element. In some embodiments, currents produced by the agitating element are asymmetric with respect to a vertical axis of the vessel. Air bubbles may also form an asymmetric configuration of bubbles about the vertical axis.

### CROSS REFERENCE TO RELATED APPLICATIONS

The present application claims priority from U.S. provisional patent application Ser. No. 61/487,800 filed May 19, 2011.

### FIELD OF THE INVENTION

Aspects relate to systems and methods for delivering oxygen to a liquid contained within a vessel. In various embodiments, systems and methods discussed may relate to introducing oxygen to a liquid within a disposable bioreactor or a disposable fermentor.

### BACKGROUND

Oxygen is an essential component for cellular respiration in living organisms that undergo aerobic metabolism. In industrial aerobic fermentation processes, oxygen is commonly provided to organisms such as bacteria or fungus grown in a fermentation broth through air injected from a sparge ring submerged in the fermentation broth. The sparge ring is typically a round metal ring with several (e.g., hundreds) holes in it. A fermentation broth often contains various types of biomass and carbohydrates such as molasses, corn starch, sugar or corn syrup. A number of broth formulations contain vegetable oil in addition to a range of minerals and nutrients that are necessary or helpful for keeping the biomass healthy and growing.

To increase biomass production rates in fermentation processes, overall air flow into a reactor vessel may be increased, providing more oxygen availability for oxidation and/or fermentation reactions. Further, the size of the air bubbles introduced into the reaction mixture may be decreased, for example, by agitation supplied by impellers or turbines. Generating air bubbles smaller in size increases the surface area to volume ratio of the air bubbles, serving to increase the rate of oxygen mass transfer from the air bubbles for dissolution into the reaction mixture.

Alternatively, commercially available gases having oxygen of a higher concentration than the oxygen concentration in air may also be input into a reaction mixture. Gases having a high concentration of oxygen would not typically require as much volume input into a fermentor or bioreactor as compared to the volume of air that would otherwise be needed for providing the same levels of oxygen.

### SUMMARY OF THE INVENTION

Aspects discussed herein relate to systems and methods for delivering oxygen to a liquid contained within a vessel. Gas having an oxygen concentration greater than the amount of oxygen found in atmospheric air is delivered toward an agitating element of a vessel at an inner mixing region where at least part of the oxygen is dissolved in the liquid. In some cases, the agitating element mixes the liquid within the vessel and produces currents in the liquid that are asymmetric with respect to a vertical axis of the vessel. Air is delivered to the liquid at a region within the vessel that is outside of the inner mixing region and at a location away from where the oxygen is introduced. Introduction of the air may occur in such a way so as to produce a mixing effect on the liquid that is independent of mixing caused by the agitating element. Air bubbles that produce a mixing effect separate from mixing caused by the agitating element may also interact with the currents produced by the agitating element so as to enhance mixing of the liquid within the vessel.

In an illustrative embodiment, a method of delivering oxygen to a liquid is provided. The method includes providing a vessel having an agitating element located within the vessel, wherein the liquid is contained within the vessel; operating the agitating element to mix the liquid within the vessel; introducing oxygen toward the agitating element at an inner mixing region of the vessel to dissolve at least part of the oxygen in the liquid; and introducing air at a region outside of the inner mixing region and at a location away from where oxygen is introduced, the introduction of air causing a mixing effect on the liquid that is independent of mixing caused by the agitating element.

In another illustrative embodiment, a method of delivering oxygen to a liquid is provided. The method includes providing a vessel having an agitating element located within the vessel and a vertical axis, wherein the liquid is contained within the vessel; operating the agitating element to produce currents in the liquid that are asymmetric with respect to the vertical axis and to mix the liquid within the vessel; and introducing air into the vessel to cause a mixing effect on the liquid that is independent of the agitating element, wherein the mixing effect interacts with the currents produced by the agitating element to enhance mixing of the liquid within the vessel.

In a further illustrative embodiment, a system for delivering oxygen to a liquid is provided. The system includes a vessel having an agitating element located within the vessel; a first gas inlet constructed and arranged to introduce a first gas having an oxygen concentration greater than about 21% by volume of the gas toward the agitating element at an inner mixing region of the vessel; and a second gas inlet constructed and arranged to introduce a second gas at a region outside of the inner mixing region and at a location away from where the first gas inlet is arranged to introduce the first gas.

Various embodiments of the present invention provide certain advantages. Not all embodiments of the invention share the same advantages and those that do may not share them under all circumstances.

Further features and advantages of the present invention, as well as the structure of various embodiments of the present invention are described in detail below with reference to the accompanying drawings.

### BRIEF DESCRIPTION OF THE DRAWINGS

The accompanying drawings are not intended to be drawn to scale. In the drawings, identical or nearly identical components that are illustrated in various figures are represented by like numerals. For purposes of clarity, not every component may be labeled in every drawing. In the drawings:

FIG. 1 is a top view of an embodiment of an agitator and a gas injection element disposed within a vessel;

FIG. 2 is a top view of an embodiment of an agitator and another gas injection element disposed within a vessel;

FIG. 3 is a cross-sectional schematic representation of another embodiment of a vessel where gases are directly injected into a liquid contained within the vessel;

FIG. 4 is a cross-sectional schematic representation of a further embodiment of a vessel where gases are directly injected into a liquid contained within the vessel;

FIG. 5 is a cross-sectional schematic representation of a different embodiment of a vessel where gases are directly injected into a liquid contained within the vessel;

FIG. 6 is a cross-sectional schematic representation of yet another embodiment of a vessel where gases are directly injected into a liquid contained within the vessel; and

FIG. 7 is a cross-sectional schematic representation of an embodiment of a vessel where gases are directly injected into a liquid contained within the vessel.

### DETAILED DESCRIPTION

The present disclosure generally relates to delivery of oxygen into a liquid contained within a vessel. In some cases, the vessel is a reaction vessel that is disposable in nature, and/or may be, without limitation, a bioreactor or a fermentor. That is, the vessel containing liquid may be useful for growing microorganisms such as cells, bacteria, yeast, fungus or other appropriate microbes. To increase the overall oxygen content of the liquid as well as increasing the efficiency of oxygen transfer within the liquid, oxygen-rich gas may be introduced into the vessel independent from a separate air stream.

Systems and methods for directly injecting oxygen into vessels like those generally described in U.S. Pat. No. 7,048,262 entitled “Method and Apparatus for Injecting Oxygen into Fermentation Processes”; U.S. Pat. No. 6,280,996 entitled “Method of Using Oxygen to Eliminate Carbon Dioxide Poisoning in Aerobic Fermentation”; U.S. Pat. No. 5,939,313 entitled “Stationary Vortex System for Direction Injection of Supplemental Reactor Oxygen”; U.S. Pat. No. 5,798,254 entitled “Gas Driven Fermentation Method using Two Oxygen-Containing Gases”; U.S. Pat. No. 5,985,652 entitled “Gas Driven Fermentation System”; and U.S. Pat. No. 5,356,600 entitled “Oxygen Enrichment Method and System” may be used for introducing gas in accordance with aspects of embodiments described herein.

In various embodiments that may provide advantages over conventional systems and methods for oxygen injection, gas having a volume of oxygen greater than about 21%, that is, greater than the volume of oxygen in atmospheric air, is introduced to liquid contained within a vessel toward one or more agitating elements (e.g., turbine, impeller, etc.) at an inner mixing region defined by the agitating element(s). At least a portion of the introduced oxygen is dissolved into the liquid. Further, air is introduced at a region outside of the inner mixing region and at a location away from where the oxygen is introduced. The air bubbles, at least partially, produce an effect of mixing in the liquid that is separate from mixing caused from currents generated in the liquid by operating the agitating element(s). However, mixing of liquid within the vessel may be further enhanced when air bubbles having been introduced into the vessel interact with the currents produced from the agitating element(s).

In some cases, as described further below, operating the agitating element(s) produces currents for mixing the liquid that are asymmetric with respect to a vertical axis of the vessel. Asymmetric mixing produced from currents created by the agitating element(s) may, in some instances, provide for a more thorough distribution of liquid and/or oxygen throughout the vessel than mixing produced from currents that are more symmetric with respect to the vertical axis of the vessel. The mixing effect provided by air bubbles also may or may not be asymmetric with respect to the vertical axis of the vessel. In some cases, an asymmetric configuration of bubbles generated from the introduction of air may give rise to more thorough mixing of liquid and/or oxygen within the vessel as compared to a more symmetric configuration of bubbles.

Whether only one or both of the independent mixing effects provided by the agitating element(s) and the generation of air bubbles are asymmetric or symmetric with respect to the vertical axis of the vessel, for various embodiments, interaction of the air bubbles with the currents produced by the agitating element(s) results in generally enhanced mixing. That is, the overall mixing produced from both the agitating element(s) and from the air bubbles gives rise to a greater degree of mixing and dissolution of oxygen within the liquid than mixing generated from only the agitating element(s) or the air bubbles by themselves.

As discussed above, embodiments described herein may be applicable to disposable vessels, such as disposable bioreactors and disposable fermentors. Because of their disposable nature, disposable bioreactors and disposable fermentors are generally smaller than traditional industrial-sized bioreactors and fermentors and, when in use, will have comparably greater cell/organism batch densities.

In some embodiments, a disposable vessel may include a disposable process bag defining a sterile environment and including a number of disposable parts (e.g., probes, sensors, mixing components such as impellers, etc.). A disposable process bag may include a flexible bag, made, for example, out of plastic, that is suitably positioned along a support structure. The bag may provide a housing for biomaterials and various components for carrying out different aspects of the process reaction, such as mixing, adding nutrients, sensing certain parameters, injecting air and/or injecting oxygen. The disposable vessel may include any suitable inlet/outlet port(s) for connecting the vessel to suitable input or output sources. For example, upon installment of the disposable vessel within a support structure, one or more appropriate gas input supplies are coupled to one or more corresponding gas inlet ports within the vessel. Additionally, a sensor included within the disposable vessel (e.g., a dissolved oxygen or dissolved carbon dioxide sensor) may be coupled to an external monitoring device once the disposable vessel is installed into the support structure.

In some embodiments, a disposable vessel is constructed to include a single-use impeller affixed to a lower portion of a flexible plastic bag. The impeller may include an impeller hub mounted on to a post where the impeller hub has an impeller blade suitable for creating currents upon rotation of the impeller. The impeller hub may be coupled to the shaft of a motor that may be provided exterior to or within the support structure of the disposable vessel. In some embodiments, the flexible plastic bag is adapted to be mounted within the support structure such that the motor comes into an aligned arrangement with the impeller hub so that the motor located exterior to the bag may drive the impeller hub as a shaft of the motor rotates. The flexible plastic bag may also have connection ports for coupling various components within the bag (e.g., sensors, inlet/outlet ports, etc.) to suitable devices that enable such components to function properly. In some embodiments, components within a disposable vessel may be coupled to processing devices exterior to the vessel via a suitable magnetic coupling arrangement.

In addition, as described herein, an oxygen-rich gas is a gas that has a concentration of oxygen that is greater than the concentration of oxygen found in air. That is, the volume of oxygen in an oxygen-rich gas is greater than about 21%. Oxygen-rich gases may have oxygen having volumes of greater than, for example, 40%, 60%, 80%, or 90% of the gas. In some cases, an oxygen-rich gas may include a gas having a high oxygen purity, such as having oxygen with a concentration of greater than 99% of the gas.

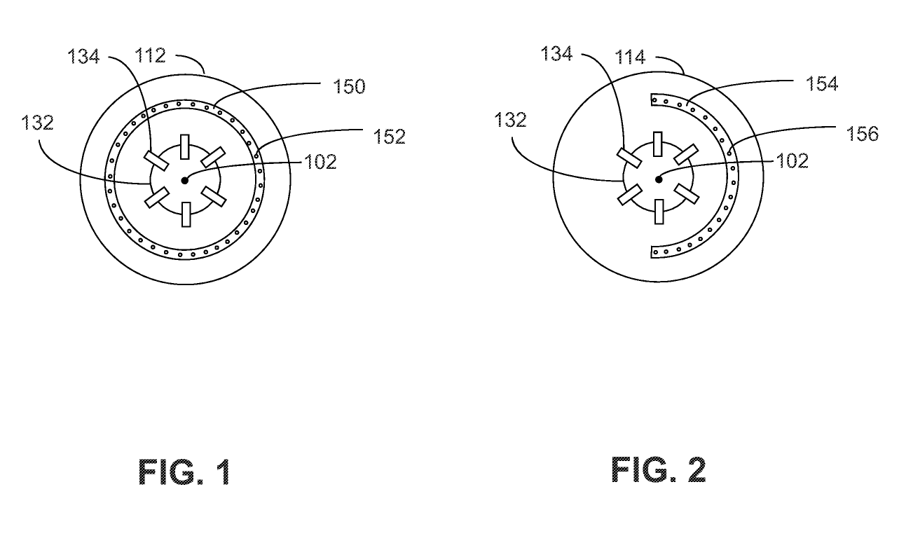

FIGS. 1 and 2 illustrate a top view of the inside of embodiments of suitable vessels. In particular, as meant to be non-limiting, these embodiments show spargers having different shapes.

In FIG. 1, a vessel 112 includes a full sparger ring 150 having holes 152 disposed within the sparger ring 150, and an agitating element 132 having six blades 134. In the full sparger ring 150, gas exits vertically from the holes 152, forming bubbles in accordance with the size of the holes and the velocity at which the gas exits the holes. That is, gas traveling at a very fast velocity will generally result in the formation of smaller sized bubbles in the liquid while gas traveling at a slower velocity will result in the formation of larger sized bubbles in the liquid. Further, the smaller the hole size, the smaller the bubbles that will be formed from gas exiting the sparger through the holes. Similarly, when holes disposed within the sparger are larger in size, the bubbles formed from gas exiting the larger holes will also be larger in size. Bubbles arising from the full sparger ring 150 form a configuration having a shape that will generally be symmetric with respect to the vertical axis 102 of the vessel 112. In some embodiments illustrated below, a full sparger ring is included which generates bubbles giving rise to a configuration of bubbles having a shape that is symmetric about the vertical axis 102.

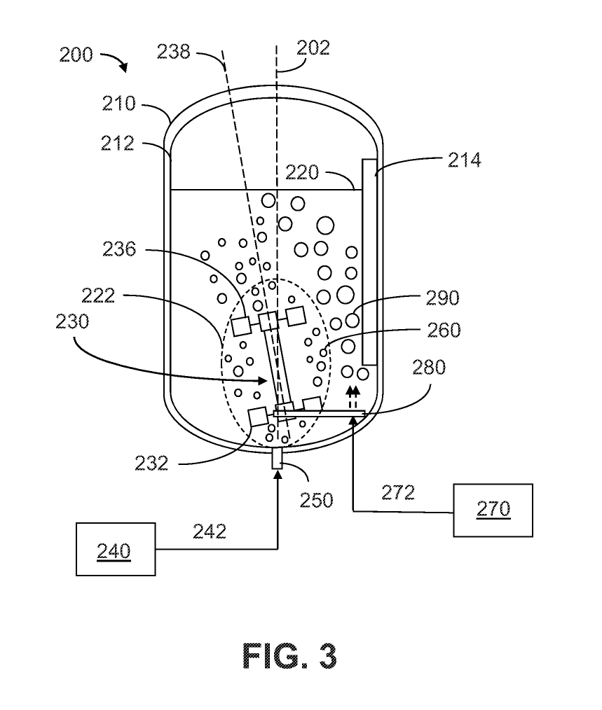

FIG. 2 depicts a vessel 114 having a half sparger ring 154 including vertically directed holes 156, and an agitating element 132 with six blades 134. Because the half sparger ring 154 is only present on one side of the vessel 114, bubbles generated by the half sparger ring 154 give rise to a configuration of bubbles that will be asymmetric with respect to the vertical axis 102 of the vessel 114. Embodiments illustrated in FIGS. 3, 4 and 5 include a half sparger ring where bubbles produced from the half sparger ring generate a configuration of bubbles that has a shape that is not symmetric with respect to the vertical axis 102.

Embodiments of vessels described herein may employ any suitable sparger having any appropriate shape or configuration for releasing gas into a liquid within a vessel. For instance, spargers not having a full-ring or half-ring configuration may be employed, such as, but not limited to, spargers with a quarter-ring, third-ring, two thirds-ring or three quarters-ring. Indeed, spargers that are not formed as a ring or a portion of a ring may also be suitable. Holes in spargers are also not limited in their shape or arrangement. For example, any number of sparger holes may be oriented in a vertical and/or a non-vertical direction so as to suitably introduce a gas into a liquid contained within the vessel. Sparger holes may be larger or smaller, depending on the desirability of a gas to exit the sparger through a larger or smaller sized hole (e.g., to form large or small sized bubbles). In some embodiments, vessels employ a membrane sparger. In such cases, gas is directed toward a membrane cover positioned about a conduit where orifices are scattered throughout the cover of the membrane sparger. Gas bubbles are formed from gas traveling through the orifices of the membrane sparger. As a result, gas entering through a membrane sparger and into a liquid are introduced as bubbles. It can be appreciated that the size and amount of bubbles generated from a membrane sparger may be suitably tailored based on the construction of the membrane sparger (e.g., size of the orifices) and the velocity of gas running through the membrane sparger.

Although the agitating element 132 includes six blades 134, the shape, configuration and arrangement of components on the agitating element are not necessary for suitable embodiments. Indeed, it is not necessary for agitating elements to include blades. For example, agitating elements may include panels having any appropriate geometry or construction for generating mixing currents. Agitating elements may comprise any suitable device for agitating a mixture, for example, an impeller such as a Ruston turbine or a pitched blade turbine. For example, the agitation system 230 of FIG. 3 includes a Ruston turbine impeller for each of first and second agitating elements 232, 236.

Agitation systems described may incorporate any appropriate configuration of agitating elements. Agitating elements included in an agitation system need not be the same. For example, in an embodiment not explicitly illustrated, a Ruston turbine may be employed as a first agitating element and a pitched blade turbine may be used as a second agitating element, or vice versa. Any number of agitating elements may be used in an agitation system, for example, one, two, three, four, or even more agitating elements. Further, agitating elements may share the same shaft and, in some cases, are operated in concert. Though, it can be appreciated that suitable agitating elements can be operated independently, and indeed, may incorporate separate shafts, or might not incorporate a shaft at all. A suitable agitating element may include a paddle that moves back and forth without rotation, or alternatively, a transducer that emits sonic energy into a mixing region. In some embodiments, an agitating element may have a surface that is constructed in a manner such that gas injected toward the agitating element collects behind the surface and is subsequently ejected from the center of the agitating element outward.

FIG. 3 illustrates an embodiment of a system 200 including a vessel 212 located within a support tank 210 and containing a liquid 220. In some embodiments, the vessel 212 is a disposable process bag lining the inside of the support tank 210. The vessel 212 includes an agitation system 230 having a first agitating element 232 and a second agitating element 236. The agitating elements 232, 236 have an axis of rotation 238 that is tilted so as to form an angle of approximately 15 degrees with the vertical axis 202 of the system 100. Gases are supplied to the liquid 220 contained within the vessel 212 through an inlet port 250 and a half sparger ring 280. In this embodiment, gas bubbles 260 originating from the inlet port 250 contain an oxygen-rich gas. A gas source 240 supplies the oxygen-rich gas through a conduit 242 (e.g., pipe or tube) and to an inlet port 250, forming the oxygen-rich gas bubbles 260 within the liquid 220. Another gas source 270 supplies air through conduit 272 (e.g., pipe or tube) and to the half sparger ring 280 to form air bubbles 290 within the liquid 220. As illustrated in FIG. 3, bubbles of the oxygen-rich gas 260 are generally smaller than air bubbles 290 having a high interfacial surface area facilitating dissolution of the oxygen-rich gas bubbles 260 into the liquid 220.

The inlet port 250 through which oxygen-rich gas enters into the vessel is positioned to introduce the oxygen-rich gas directly toward the agitation system 230 at an inner mixing region 222 of the vessel 212. The inner mixing region 222, as considered herein and as schematically depicted in FIG. 3, is a zone in the immediate vicinity where the agitation system is located. As shown, the inlet port 250 is located below the first agitating element 232 and is oriented to deliver the oxygen-rich gas directly toward the first agitating element 232. As oxygen-rich gas is injected from the inlet port 250 directly into the space occupied by the first agitating element 232, or the zone in the immediate vicinity where the agitation system is located, the oxygen-rich gas is introduced into inner mixing region 222.

Conversely, air bubbles 290, are delivered to the liquid 220 via the half sparger ring 280 at a location outside of the inner mixing region 222 away from where the oxygen-rich gas is delivered. Introduction of air through the half sparger ring 280 produces a mixing effect on the liquid 220. The air bubbles 290 are of a large size such that a sufficient drag is induced on the liquid so as to cause mixing. The smaller oxygen-rich bubbles 260, in contrast, are not as large as the air bubbles 290 and so do not generate sufficient movement of the liquid for mixing to occur. As illustrated, the air bubbles 290 travel through the liquid up along the outer edge of the vessel 212 and around the inner mixing region 222 of the vessel 212, producing an asymmetric mixing effect with respect to the vertical axis 202. In some embodiments, and as depicted FIG. 3, the vessel 212 also includes a baffle 214 so as to further direct air bubbles in a manner for producing even more of an asymmetric mixing effect. The mixing effect produced from the air bubbles 290 provides generally for improved oxygen dissolution within the liquid. As a result, with greater oxygen availability for organisms disposed within the liquid, reaction and/or fermentation processes occurring in the vessel are also improved and more efficient. Such a mixing effect is independent of the mixing caused by currents produced from operating the agitating elements 232, 236.

When running the agitating elements 232, 236, the tilt of the axis of rotation 238 of approximately 15 degrees with the vertical axis 202 produces currents that are asymmetric with respect to the vertical axis 202. As illustrated, the oxygen-rich gas bubbles 260 travel along currents produced by the agitating elements 232, 236 in an asymmetric pattern. It can be appreciated that agitating elements can be oriented and/or configured in any suitable manner so as to produce appropriately asymmetric currents. For example, agitating elements may be tilted so that the axis of rotation with respect to the vertical axis forms an angle greater than 0 degrees, such as greater than 5 degrees, 10 degrees, 20 degrees, 30 degrees, or more. Alternatively, agitating elements are not required to be tilted with respect to the vertical axis at all. Indeed, agitating elements may be intrinsically structured so as to produce asymmetric currents. In some embodiments, the blades of an impeller may be shaped, oriented or positioned in a manner that causes currents to be formed that are asymmetric with respect to the vertical axis. Similar to the mixing effect produced from the air bubbles 290, mixing brought about by currents generated from the agitating elements provides for improved oxygen dissolution within the liquid. Accordingly, oxygen input may be more efficiently utilized and reaction and/or fermentation processes within the vessel may also be improved.

Although mixing effects generated by operation of the agitating elements and movement of the air bubbles introduced by the sparger are independent from one another, the mixing effect produced by movement of the air bubbles may interact with the mixing provided by the agitating elements so as to further enhance mixing of the liquid 220 within the vessel 212. That is, the combined mixing effect of the interaction between air bubble movement and the currents generated from the agitating elements further improves oxygen dissolution and reaction/fermentation processes within the liquid. Thus, less oxygen input may be required for a reaction and/or fermentation process within the vessel with combined mixing as compared for the same result in a reaction and/or fermentation process without such combined mixing. In some cases, mixing induced by agitating elements and/or movement of air bubbles that is asymmetric, as described above, may enhance oxygen dissolution more than that if the mixing were symmetric with respect to the vertical axis.

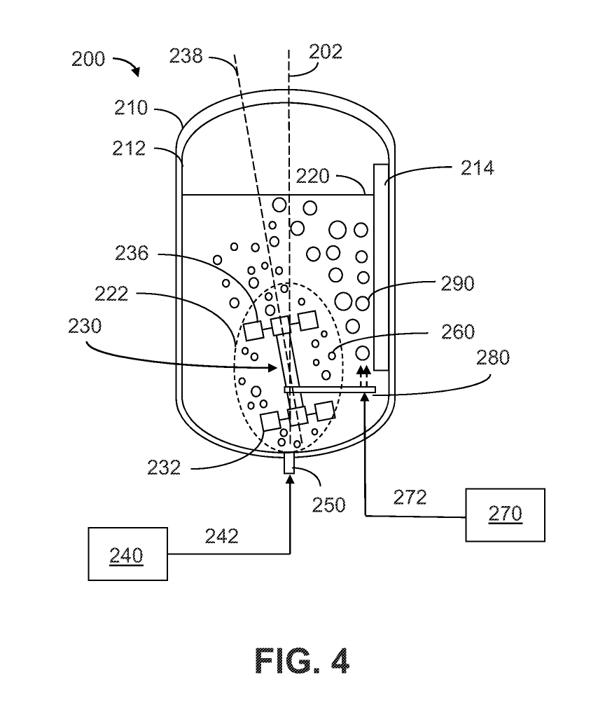

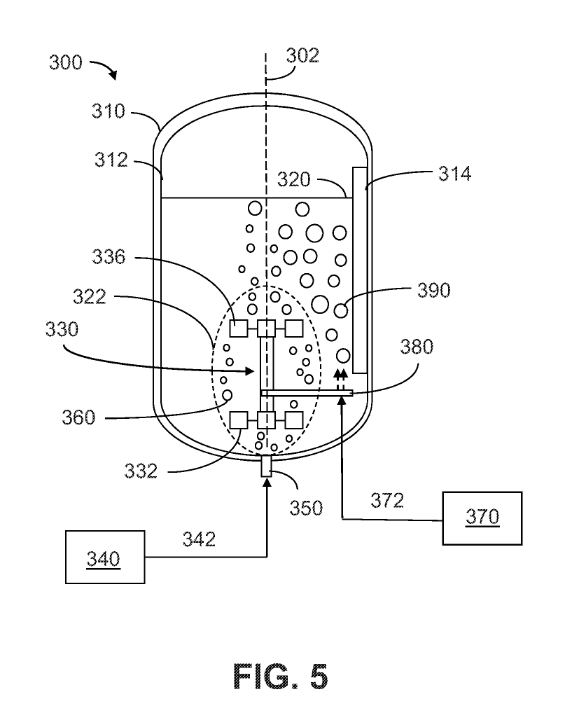

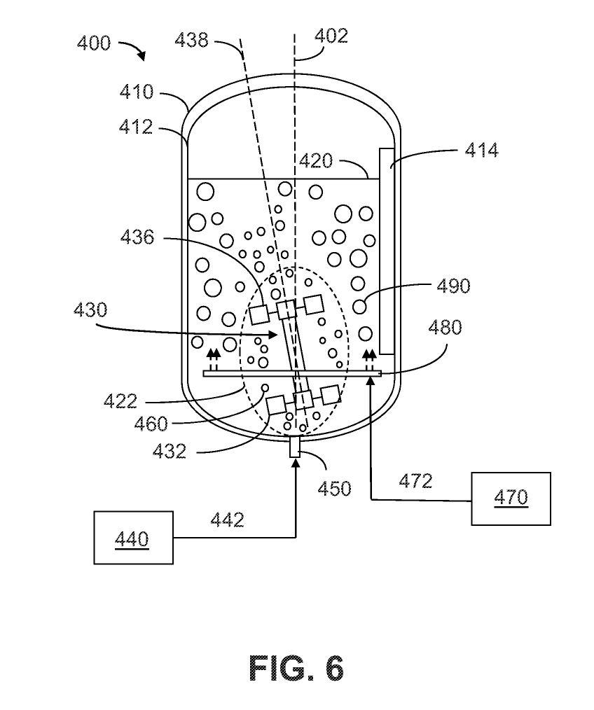

Any suitable modification can be made to the vessel so as to produce an appropriate result. For example, the position of the sparger can be altered, as depicted in FIG. 4. Or, the agitating elements are not required to be tilted, as shown in FIG. 5. Alternatively, as illustrated in FIG. 6, a tilted set of agitating elements may be employed in conjunction with a full sparger ring.

FIG. 4 depicts an illustrative embodiment of a configuration of the system 200 where the vessel 212 is altered such that the half ring sparger 280 is positioned at a higher position than that of the implementation shown in FIG. 3. In some cases, it may be beneficial to introduce air bubbles 290 at a location further above the bottom of the vessel, for example, so as to minimize any risk of co-mingling of air bubbles with oxygen-rich gas located within the inner mixing region 222. By positioning the sparger 280 at a higher position, the air bubbles 290 are still able to produce a mixing effect as that described above, yet coalescing of oxygen-rich gas with the air bubbles is less likely. When small bubbles of oxygen-rich gas are combined with larger air bubbles, oxygen dissolution into the liquid generally takes a longer time to occur. Thus, by reducing any opportunity for oxygen-rich gas bubbles 260 to coalesce with larger air bubbles 290, oxygen availability to organisms disposed in the vessel is generally increased. Therefore, less oxygen input and potentially power input (e.g., in operating the agitation system) may be required for reaction and/or fermentation. In various embodiments, the vessel 212 may be a disposable vessel suitable for use as a bioreactor and/or a fermentor.

FIG. 5 depicts another illustrative embodiment of a system 300 having a vessel 312 where the agitation system 330 is not tilted with respect to the vertical axis 302, yet air is introduced through a half sparger ring 380. Agitating elements 332, 336 have an axis of rotation that is substantially coincident with the vertical axis 302. Oxygen-rich gas bubbles 360 are supplied toward the agitation system 330 at the inner mixing region 322 by inlet port 350 via corresponding gas source 340 and conduit 342. Air bubbles 390 are generated from the half sparger ring 380 having been supplied by gas source 370 via conduit 372. As illustrated above, the air bubbles 390 are introduced into the liquid 320 outside of the inner mixing region 322 at a location away from where oxygen-rich gas is introduced, so as to prevent substantial coalescing of the oxygen-rich gas bubbles with the air bubbles.

Similar to embodiments described above, movement of the air bubbles delivered through the half sparger ring 380 produces a mixing effect on the liquid 320 that is independent from mixing provided from currents generated by operating the agitating elements 332, 336. The air bubbles 390 travel up through the liquid along the outer edge of the vessel 312 and around the inner mixing region 322, giving rise to an asymmetric mixing effect with respect to the vertical axis 302. The vessel 312 also includes a baffle 314 that is employed to direct the movement of air bubbles for an even greater asymmetric mixing effect.

Operation of agitating elements 332, 336 produces currents that are generally symmetric with respect to the vertical axis 302 of the vessel 312. The oxygen-rich gas bubbles 360 travel along such currents in a manner that encourages oxygen within the bubbles to dissolve in the liquid 320.

The mixing effects provided by operating the agitating elements 332, 336 and introducing the air bubbles by the sparger 380 are independent from one another. Though, the air bubbles from the sparger may interact with currents produced from the agitating elements in manner that further enhances mixing of the liquid 320, hence, oxygen dissolution and reaction/fermentation processes within the liquid are improved. The vessel 312 may be a disposable vessel, such as a disposable bioreactor or a disposable fermentor where components of the disposable vessel may be coupled to a support structure and suitable components for furthering a desired reaction/fermentation process.

FIG. 6 shows yet another illustrative embodiment of a system 400 having a vessel 412 where air is introduced through a full sparger ring 480, in contrast to a half sparger ring, and the agitation system 430 is tilted with respect to the vertical axis 402. In this embodiment, agitating elements 432, 436 have an axis of rotation 438 that forms an angle of approximately 15 degrees with respect to the vertical axis 402. Similar to embodiments described above, oxygen-rich gas bubbles 460 are introduced toward the agitation system 430 at the inner mixing region 422 via inlet port 450 having been supplied from gas source 440 via conduit 442. Air bubbles 490 are produced from the full sparger ring 480 supplied by gas source 470 via conduit 472. The vessel 412 includes a baffle 414 for directing air bubbles in a manner that assists mixing of the liquid 420 so that oxygen is more easily dissolved into the liquid. Additionally, air bubbles 490 are introduced into the liquid 420 outside of the inner mixing region 422 at a location away from the inlet port 450 where the oxygen-rich gas is introduced.

The tilt of the axis of rotation 438 of approximately 15 degrees with the vertical axis 402 gives rise to asymmetric currents about the vertical axis 202 when agitating elements 432, 436 are operated. Mixing provided by such asymmetric currents generated from the agitating elements may give rise to improved dissolution of the oxygen-rich gas within the liquid.

Movement of air bubbles introduced to the liquid 420 through the full sparger ring 480 provides a mixing effect on the liquid 420 which is independent from mixing effects produced from currents generated by running agitating elements 432, 436. However, air bubbles delivered via the full ring sparger 480 may interact with currents produced from the agitating elements 432, 436 to enhance mixing of the liquid 420 so as to improve oxygen dissolution and reaction/fermentation processes within the liquid. It can be appreciated that the vessel 412 may be a disposable vessel, such as a disposable bioreactor or a disposable fermentor, that can be suitably coupled to a support structure and devices associated therewith.

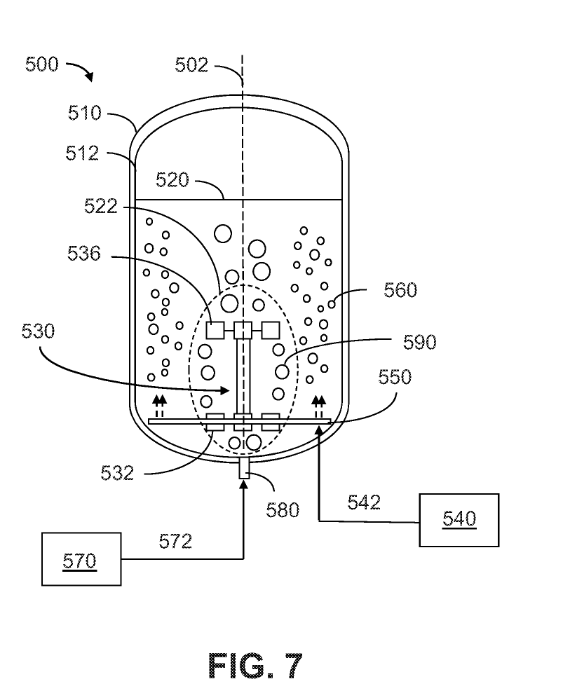

Systems and methods described for introducing gas into a disposable vessel may provide for any suitable alternative arrangement for delivering gas. For example, in some embodiments, air may be introduced into a disposable vessel at an inner mixing region and oxygen may be introduced into the disposable vessel at a location outside of the inner mixing region. FIG. 7 depicts an embodiment of a system 500 where a vessel 512 containing a liquid 520 is disposed within a support tank 510. In some embodiments, the vessel 512 is disposable, for example, the vessel may be a disposable bioreactor or a disposable fermentor. As such, the vessel may include a thin plastic bag that lines the walls of the support tank 510 with the vessel also having a number of components that are useful for running a reaction and/or creating a suitable environment for organisms to grow. Located inside the vessel 512 is an agitation system 530 that includes a first agitating element 532 and a second agitating element 536. The agitating elements 532, 536 have an axis of rotation that is substantially coincident with the vertical axis 502 of the system 500. Gas is supplied to the liquid 520 within the vessel 512 through a sparger ring 550 and an inlet port 580. In this embodiment, sparger ring 550 provides bubbles 560 to the liquid containing an oxygen-rich gas. A gas source 540 supplies the oxygen-rich gas through a conduit 542, such as a pipe, to the sparger ring 550. Additionally, gas source 570 supplies air through a conduit 572, such as a pipe, leading to the inlet port 580 so as to form air bubbles 590 in the liquid.

The inlet port 580 from which air bubbles 590 are formed is arranged so as to introduce the air directly toward the agitation system 530 at an inner mixing region 522 of the vessel 512. As air is injected directly toward the first agitating element 532 from the inlet port 580, the air is considered to be introduced into the inner mixing region 522. Gas bubbles 560 of oxygen-rich gas are introduced via sparger ring 550 at a location outside of the inner mixing region 522. Introducing oxygen at locations away from where air is introduced is generally helpful to minimize substantial coalescing of the oxygen-rich gas bubbles with the air bubbles, as coalescing of the bubbles can reduce oxygen dissolution into the liquid.

In some cases, and as depicted in FIG. 7, gas bubbles 560 of oxygen-rich gas can, on average, be smaller than air bubbles 590. As bubbles of oxygen-rich gas are generally smaller than the air bubbles, the average interfacial surface area of the oxygen-rich gas bubbles is greater than the average interfacial surface area of the air bubbles. Thus, when the oxygen-rich bubbles are smaller than the air bubbles, the rate of oxygen dissolution into the liquid from the oxygen-rich bubbles is greater than the rate at which oxygen from the air bubbles dissolves into the liquid.

As the rotational axis of the agitation system 530 including the agitating elements 532, 536 is substantially coincident with the vertical axis 502 of the system 500, operating the agitating elements 532, 536 produces currents in the liquid that are generally symmetric with respect to the vertical axis 502. Accordingly, air bubbles 590 injected into the inner mixing region 522 and mixed by currents generated by the agitating elements 532, 536 will form a configuration of bubbles that is symmetric with respect to the vertical axis 502. Since sparger ring 550 is also symmetric about the vertical axis 502, bubbles 560 of oxygen-rich gas generally form a symmetric configuration of bubbles with respect to the vertical axis 502. The configuration of air bubbles 590 and oxygen-rich gas bubbles 560 exhibit a generally radial symmetry.

Aspects described herein may be employed in any appropriate oxidation and/or fermentation reaction mixture. For example, in fermentation reactions, the reaction mixture may include a fermentation broth that generally includes water, nutrients or fermentable constituents such as corn syrup, molasses and glucose, and a biological organism such as bacteria, fungus and/or yeast. Fermentation mixtures may contain additives such as antifoam agents, nitrates or chemicals for pH adjustment, and the like. Some examples of fermentation products that may be produced by methods described herein include antibiotics such as penicillin, erythromycin and tetracycline; organic chemicals such as ethanol, sorbitol and citronellol; organic acids such as citric acid, tartaric acid and lactic acid; amino acids such as L-lysine and monosodium glutamate; polysaccharides such as baker's yeast and xanthan gum; vitamins such as ascorbic acid and riboflavin; and other products including enzymes, insecticides, alkaloids, hormones, pigments, steroids, vaccines, interferon and insulin. Liquid phase oxidation reactions may also be carried out using methods described. For example, such reactions may include the oxidation of toluene to benzoic acid, the oxidation of p-xylene to p-toluic acid, the production of hydrogen peroxide through the oxidation of hydroquinone, the oxidation of toluene to phenol, and the oxidation of paraxylene to terephthalic acid.

Embodiments described herein also provide for more efficient delivery of oxygen to living organisms within process vessels, such as disposable fermentors.

In general, industrial sized fermentors may be between 40 and 200 times larger than disposable sized fermentors. For example, an industrial fermentor may have a volume of about 200,000 L or greater whereas a disposable fermentor may have a volume ranging between about 1,000 and 5,000 L. As the volume of disposable fermentors are typically much smaller than traditional fermentors, the biomass within disposable fermentors, comparatively, can be denser and more than 50% greater, placing limitations on nutrients and oxygen supply. However, it should be appreciated that systems and methods described herein are applicable to large scale vessels, such as industrial sized fermentors, with advantageous results.

When systems and methods described are implemented for disposable bioreactors or disposable fermentors, which have high cell/organism batch densities, oxygen consumption and/or power requirements may be reduced. In some embodiments, for high density batches of cells/organisms typically found in disposable fermentors, high levels are recorded for oxygen transfer efficiency, effectively reducing oxygen consumption and/or power requirements within vessel.

For purposes described herein, oxygen transfer efficiency (OTE) is measured as the amount of oxygen consumed in a vessel (e.g., reactor or fermentor) compared to the oxygen input into the vessel. For example, a 25% measure of air OTE would indicate that a quarter of the oxygen available through input of air into the vessel had been consumed by the process (e.g., reaction or fermentation) occurring within the vessel. For instance, oxygen consumption for disposable fermentors may be reduced by as much as 30% to 40% or more when embodiments described herein are employed for oxygen injection into the fermentation broth of a disposable vessel.

Various gas parameters were measured for disposable fermentors using oxygen injection systems and methods described herein as compared with a conventional gas injection system.

In a Conventional Example, for mixing a fermentation broth in a disposable fermentor, a Ruston turbine was used as a lower agitating element and a pitched blade turbine was used as an upper agitating element. Air was injected at an inner mixing region from the bottom of the fermentor directly toward the Ruston turbine, and oxygen-rich gas was injected outside of the inner mixing region via a full-ring sparger.

In Example 1, a system and method was employed for oxygen and air injection into a disposable fermentor similar to the embodiment depicted in FIG. 7 where both agitating elements in the agitation system are Ruston turbines. In addition, air was injected at an inner mixing region directly toward the lower Ruston turbine, and oxygen-rich gas was injected outside of the inner mixing region via a full-ring sparger.

In Example 2, a system and method for oxygen and air injection into a disposable fermentor described and illustrated in FIG. 3 was employed where the agitation system is tilted and a half-ring sparger is employed. Further, oxygen-rich gas was injected at an inner mixing region from the bottom of the disposable fermentor directly toward the lower Ruston turbine, and air was injected outside of the inner mixing region through the half-ring sparger. The results of a number of examples used for the disposable fermentors are provided in Table 1 below.

TABLE 1

| | Conventional | | |
|---|---|---|---|
| | Example | Example 1 | Example 2 |
| Working Volume (L) | 200 | 200 | 200 |
| Air sparge (vvm) | 0.55 | 0.55 | 0.55 |
| O₂ sparge (vvm) | 0.25 | 0.17 | 0.15 |
| O₂ transfer rate | 254 | 254 | 254 |
| (mmol O₂/L/h) | | | |
| OTE of air (%) | 21.3% | 21.3% | 12.8% |
| OTE of O₂ -rich gas (%) | 28.1% | 42.1% | 52.6% |
| Overall OTE (%) | 25.9% | 33.6% | 31.7% |

In these examples, for the same oxygen transfer rate (OTR), less oxygen input is required to reach a similar overall fermentation reaction result. That is, the overall OTE for Examples 1 and 2 was greater as compared to the Conventional Example, indicating that oxygen dissolution occurred more readily for Examples 1 and 2 for both air and oxygen-rich gas inputs. However, the implementations of Examples 1 and 2 appeared to be distinctly more advantageous than the implementation of the Conventional Example as measured by OTE with respect to the injection of oxygen-rich gas. In addition, though not explicitly recorded, the power consumption requirements for embodiments incorporating systems and methods of oxygen injection described herein may be less than power consumption requirements for conventional systems and methods.

As noted above, the addition of oxygen-rich gas to liquid contained in a vessel occurs in a manner that does not substantially mix with air bubbles injected into the liquid, resulting in higher dissolved oxygen content liquids and higher oxygen utilization efficiency than would have been obtained if the additional oxygen-rich gas had been added with the air stream, or such that the oxygen would combine substantially with the air. As also discussed, aspects described may be employed to enhance concentrations of dissolved oxygen for fermentation systems, as well as in organic oxidation processes, such as those occurring in bioreactor systems. Suitable methods for gas injection include but are not limited to supersonic gas injection nozzles, simple pipes or gas spargers, orifices, venturi type nozzles or gas-liquid nozzles, depending on the requirements of a given application. If a high oxygen content liquid, or liquid plus oxygen gas is used, the injection device for oxygen addition may be simple pipes, spargers, venturi nozzles or gas-liquid nozzles as desired for the particular application.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the scope of the invention. Accordingly, the foregoing description and drawings are by way of example only.

What is claimed is:

1. A method of delivering oxygen to a liquid, the method comprising:
providing a disposable bioreactor or a disposable fermentor having an agitating element located therein, wherein the liquid is contained within the disposable bioreactor or a disposable fermentor;
operating the agitating element to mix the liquid within the disposable bioreactor or a disposable fermentor;
introducing oxygen toward the agitating element at an inner mixing region of the disposable bioreactor or a disposable fermentor to dissolve at least part of the oxygen in the liquid; and
introducing air at a region outside of the inner mixing region and at a location away from where oxygen is introduced, the introduction of air causing a mixing effect on the liquid that is independent of mixing caused by the agitating element.

2. The method of delivering oxygen to a liquid of claim 1, wherein the agitating element comprises at least one Ruston turbine or pitched blade turbine

3. The method of delivering oxygen to a liquid of claim 1, wherein introducing oxygen toward the agitating element comprises introducing oxygen at a region below the agitating element.

4. The method of delivering oxygen to a liquid of claim 1, wherein introducing oxygen toward the agitating element comprises introducing oxygen through a gas inlet pipe.

5. The method of delivering oxygen to a liquid of claim 1, wherein introducing air at the region outside of the inner mixing region comprises introducing air through a sparger, and wherein the sparger comprises a half-ring sparger or a membrane sparger.

6. The method of delivering oxygen to a liquid of claim 1, wherein operating the agitating element produces currents in the liquid that are asymmetric with respect to a vertical axis of the disposable bioreactor or disposable fermentor.

7. The method of delivering oxygen to a liquid of claim 6, wherein the mixing effect caused by the introduction of air interacts with the currents produced by the agitating element to enhance mixing of the liquid within the disposable bioreactor or disposable fermentor.

8. The method of delivering oxygen to a liquid of claim 1, wherein the disposable bioreactor or a disposable fermentor comprises a disposable bag lining an inner wall of a support structure.

9. A method of delivering oxygen to a liquid, the method comprising:
providing a disposable bioreactor or a disposable fermentor having an agitating element located therein and a vertical axis, wherein the liquid is contained within a disposable bioreactor or a disposable fermentor;
operating the agitating element to produce currents in the liquid that are asymmetric with respect to the vertical axis and to mix the liquid within the disposable bioreactor or disposable fermentor; and
introducing air into the disposable bioreactor or disposable fermentor to cause a mixing effect on the liquid that is independent of the agitating element, wherein the mixing effect interacts with the currents produced by the agitating element to enhance mixing of the liquid within the disposable bioreactor or disposable fermentor.

10. The method of delivering oxygen to a liquid of claim 9, further comprising introducing oxygen toward the agitating element at an inner mixing region of the disposable bioreactor or disposable fermentor to dissolve at least part of the oxygen in the liquid.

11. The method of delivering oxygen to a liquid of claim 9, wherein the agitating element has an axis of rotation forming an angle greater than 0 degrees with respect to the vertical axis.

12. The method of delivering oxygen to a liquid of claim 9, wherein the agitating element comprises at least one Ruston turbine or pitched blade turbine.

13. The method of delivering oxygen to a liquid of claim 9, wherein introducing air into the vessel comprises introducing air at a region outside of the inner mixing region and at a location away from where oxygen is introduced.

14. The method of delivering oxygen to a liquid of claim 9, wherein introducing air into the vessel comprises introducing air through a sparger, wherein the sparger comprises a half-ring sparger or a membrane sparger.

15. The method of delivering oxygen to a liquid of claim 9 wherein the disposable bioreactor or a disposable fermentor comprises a disposable bag lining an inner wall of a support structure.

16. A system for delivering oxygen to a liquid, the system comprising:
a disposable bioreactor or a disposable fermentor having an agitating element located within the disposable bioreactor or disposable fermentor;
a first gas inlet constructed and arranged to introduce a first gas having an oxygen concentration greater than about 21% by volume of the gas toward the agitating element at an inner mixing region of the disposable bioreactor or disposable fermentor; and
a second gas inlet constructed and arranged to introduce a second gas at a region outside of the inner mixing region and at a location away from where the first gas inlet is arranged to introduce the first gas.

17. The system for delivering oxygen to a liquid of claim 16, wherein the agitating element has an axis of rotation forming an angle greater than 0 degrees with respect to a vertical axis.

18. The system for delivering oxygen to a liquid of claim 16, wherein the agitating element comprises at least one impeller comprising a Ruston turbine or a pitched blade turbine.

19. The system for delivering oxygen to a liquid of claim 16, wherein the first gas inlet is constructed and arranged to introduce the first gas at a region below the agitating element.

20. The system for delivering oxygen to a liquid of claim 16, wherein the second gas inlet comprises a sparger, wherein the sparger comprises a half-ring sparger or a membrane sparger.
